(19) United States
(12) Patent Application Publication
(10) Pub. No.: US 2005/0143828 A1
(43) Pub. Date: Jun. 30, 2005

(54) Sleeve and method for use with modular orthopaedic implants

(76) Inventors: Michael S. Collins, Warsaw, IN (US); Laban D. Cook, Claypool, IN (US)

Correspondence Address: ZIMMER TECHNOLOGY - REEVES, P. O. BOX 1268, ALEDO, TX, 76008, US

(21) Appl. No.: 10/749,483

(22) Filed: Dec. 31, 2003

(51) Int. Cl. A61F 2/30

(52) U.S. Cl. 623/18.11

(57) ABSTRACT

A sleeve and method for use with modular orthopaedic implants is provided.

### FIELD OF THE INVENTION

The invention relates to a sleeve for use with modular orthopaedic implants.

### BACKGROUND

Medical implants to replace or augment various parts of the mammalian body have been successfully used to reduce pain and improve function. For example, orthopaedic implants for replacing portions of bones and joints damaged by disease and/or trauma often eliminate pain and/or increase mobility. Orthopaedic implants for hips, knees, shoulders, ankles, elbows, wrists, the digits of the hands and feet, vertebral bodies, spinal discs, and other bones and joints have been developed. Many orthopaedic implants are made more versatile by providing them as separate modular components that can be combined to form an implant suited to a particular patient's condition. Where such modular components are supplied, a means for attaching them to one another is provided such as a male/female junction. To aid the selection of the appropriate modular components, provisional, or trial fit, components are often provided for test fitting component geometries before the actual implants are implanted.

### SUMMARY

The present invention provides a sleeve and method for use with modular orthopaedic implants.

In one aspect of the invention, a sleeve includes a hollow sleeve body having an outer portion able to be received in a female junction element and an inner portion able to receive a male junction element of a modular orthopaedic implant. The sleeve includes means for temporarily maintaining the first and second components in an assembled condition.

In another aspect of the invention, a combination includes a sleeve and a modular orthopaedic implant. The implant has a first component with a male junction element and a second component with a female junction element for receiving the male junction element to couple the components together. The sleeve has an outer portion able to be received in the female junction element and an inner portion able to receive the male junction element. The combination further has means for temporarily maintaining the first and second components in an assembled condition.

In another aspect of the invention, a method of temporarily joining modular orthopaedic implant components includes providing a modular orthopaedic implant having a first component with a male junction element and a second component with a female junction element for receiving the male junction element to couple the components together; providing a sleeve having a hollow sleeve body with an outer portion able to be received in the female junction element and an inner portion able to receive the male junction element; and positioning the sleeve between the first and second components with the sleeve received in the female junction element and the male junction element received in the sleeve to temporarily maintain the first and second components in an assembled condition.

### BRIEF DESCRIPTION OF THE DRAWINGS

Various embodiments of the present invention will be discussed with reference to the appended drawings. These drawings depict only illustrative embodiments of the invention and are not to be considered limiting of its scope.

FIG. 1 is an exploded perspective view of an illustrative modular hip stem with illustrative sleeves according to the present invention;

FIGS. 2-3 are exploded side section views of the implant and sleeves of FIG. 1 showing the sleeves being applied to the implant; and

FIGS. 4-6 are exploded perspective views of a portion of the implant and one of the sleeves of FIG. 1 showing the sleeve being deployed from an alternate rolled configuration.

### DESCRIPTION OF THE ILLUSTRATED EMBODIMENTS

Embodiments of a sleeve for use with modular orthopaedic implants are applicable to a variety of implants for use throughout the body. A modular femoral hip stem has been used to illustrate the invention. However, the invention may also be applied to various other implants including orthopaedic implants for hips, knees, shoulders, ankles, elbows, wrists, the digits of the hands and feet, vertebral bodies, spinal discs, and other suitable implants. The sleeve may be used to temporarily couple provisional implant components together, to temporarily couple actual implant components to provisional implant components, and/or to temporarily couple actual implant components together. The sleeve is useful to provide a temporary friction and/or press-fit locking assembly. It is also useful to permit temporary assembly of actual implant components without requiring the use of the actual implantable locking mechanism so that the components may be uncoupled and switched for alternate sizes and/or shapes easily and without damage.

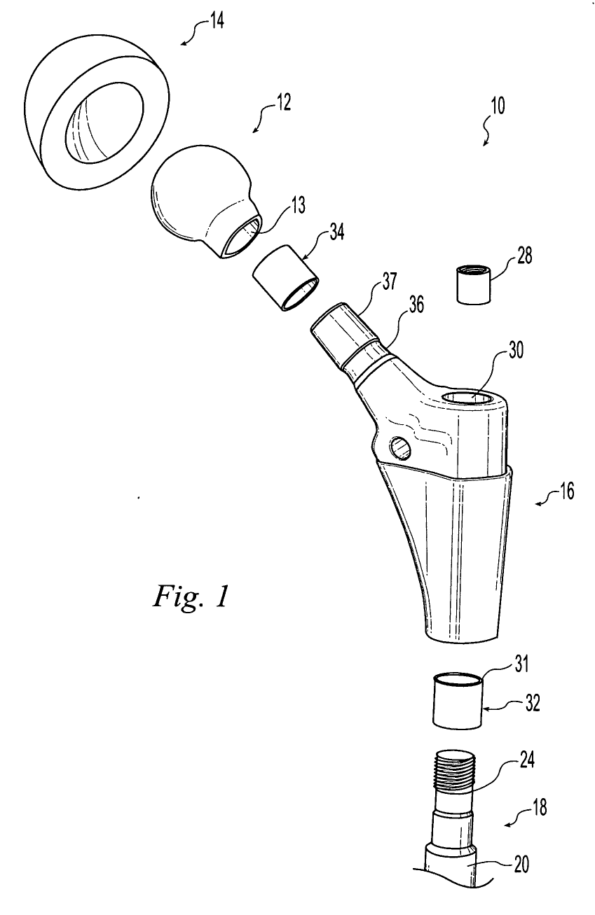

FIGS. 1-3 depict an illustrative modular femoral hip implant 10 for replacing the proximal head and neck of a femur of a hip joint. In use, the proximal head and neck are surgically removed and the femoral hip implant 10 is inserted into the proximal femur. The femoral hip implant 10 supports a femoral head 12 that may be a modular and separate component as shown. Optionally, the femoral head 12 may be integral to the femoral hip implant 10. An acetabular component 14 may be implanted in the acetabulum of the pelvis to articulate with the femoral head 12. Optionally, the femoral head 12 may articulate with the natural acetabulum.

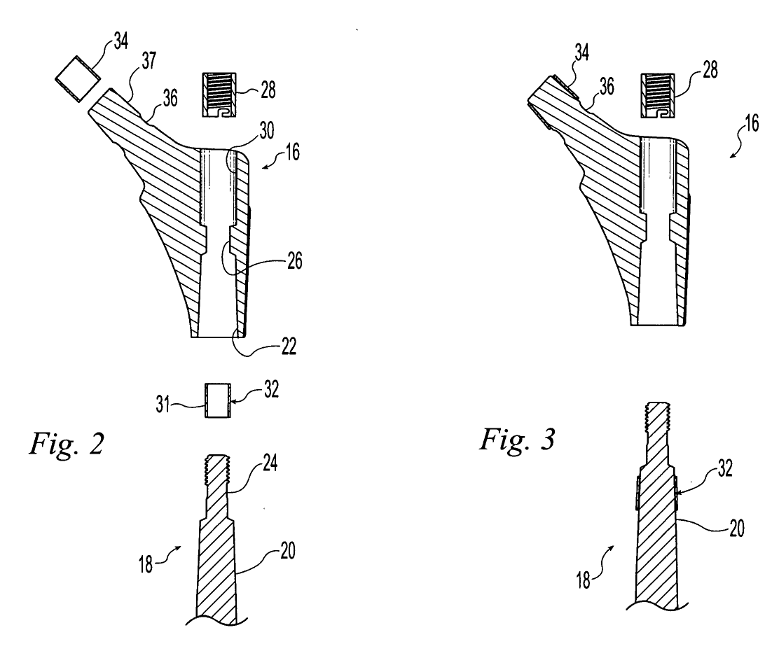

The femoral hip implant 10 may include modular components such as a proximal body 16 and a stem 18. The modular components may be provided in a variety of shapes and/or sizes to permit intraoperative assembly of an implant optimized for a particular patient's needs. The illustrative implant 10 includes a male conical taper 20 formed on the stem 18 and a corresponding female conical taper 22 (FIG. 2) formed in the proximal body 16. The gender of these components may be reversed and still be within the scope of the invention. Likewise, other shapes of the male/female couplings may be provided within the scope of the invention. For example, the male/female junction may have a cylindrical cross section, a polygonal cross section (with or without a taper), and/or other suitable shape. The stem 18 further includes a threaded stud 24 extending axially outwardly that is received by a through bore 26 in the proximal body 16 communicating with the female taper 22. A nut 28 is received by a counter bore 30 in the proximal body 16 and threads onto the stud 24 to secure the proximal body 16 and stem 18 together. The tapers 20, 22 may be self-locking tapers which may be used alone or in combination with the threaded stud 24 and nut 28.

It is desirable to trial fit proximal bodies 16 and stems 18 intraoperatively to ascertain the best fit for a particular patient without locking them together with the actual implant locking mechanism. One advantage of not using the actual locking mechanism is that the actual locking mechanism is designed to lock the components tightly together and thus it may be difficult to separate the components once they are locked. Another advantage of not using the actual locking mechanism is that repeated assembly and disassembly may cause wear, scratching, or other damage to the mechanism that might compromise its later use to actually lock the components together. Another advantage of not using the actual locking mechanism is that it may be desirable to use a provisional component made of a different material than the actual implant, for example a lightweight, inexpensive, polymer provisional. For example, a polymer proximal body provisional component 16 may be used with the actual metal stem implant 18. In this way, the stem 18 can be set once and different proximal body sizes and shapes can be tried to arrive at the best fit. By using provisional proximal bodies, there is no need to contaminate multiple actual proximal body implants during trial fitting. However, a polymer provisional proximal body 16 may not grip the stem 18 tightly enough to prevent rotation and/or axial translation of the proximal body 16 relative to the stem 18 during the trial fitting.

All of these advantages are provided and shortcomings overcome by using an intermediate sleeve 32 positioned between the tapers of the mating components to temporarily maintain the implant 16 components in an assembled arrangement. The sleeve 32 may be press fit between the components. The sleeve 32 may prevent damage to the components and may provide sufficient friction to hold the parts together for the trial fitting. In the illustrative example, the sleeve 32 prevents the proximal body 16 from rotating relative to the stem 18 and from dislocating axially from the stem 18. The sleeve 32 includes a hollow sleeve body 31 having an outer portion able to be received in the taper 22 of the proximal body 16. The sleeve body 31 has an inner portion able to receive the male taper 20 of the stem 18. The sleeve body 31 may be the same size and shape as the junction components initially or it may stretch to fit the components. Both ends of the sleeve may be open as shown, or one end of the sleeve 32 may be closed. The sleeve 32 is sandwiched between the components as the junction is pressed together into an assembled arrangement.

The sleeve 32 may be used alone or it may be used in conjunction with the nut 28. If the sleeve 32 is used with the nut 28, the nut helps to maintain the implant in the assembled arrangement while the sleeve 32 protects the junction surfaces and/or prevents the junction from locking together too tightly while providing sufficient frictional engagement to maintain the components' relative positions. For example, the junction may include self-locking Morse-type tapers. The sleeve 32 may be used to prevent the tapers from fully seating and locking. However, the sleeve 32 provides sufficient rotational and axial frictional locking to permit trial fitting.

The sleeve 32 may be made of a material with a sufficient coefficient of friction with the implant components to temporarily maintain the modular components in this assembled arrangement. The sleeve 32 may also be made of a material with sufficient resilience to permit it to stretch to a thin cross section when it is placed over the male side of the junction and/or to permit it to compress when the modular components are assembled to provide a press-fit engagement and retention of the components. The junction may have surface features and/or texturing to interdigitate with the sleeve 32 Suitable materials for the sleeve include natural and manmade rubbers and other elastomers such as latex, silicone, vinyl, isoprene, and other materials with sufficient friction and/or resilience to temporarily maintain the modular components in an assembled arrangement. The present investigators have found that a latex sleeve approximately 0.010-0.015 inches thick provides the desired function when positioned between the self-locking tapers between metal proximal body and stem components of a modular hip implant. Other thicknesses may be advantageous for other modular junctions. The sleeve 32 may be disposable or reusable. Advantageously the sleeve is provided as a disposable unit that need not be cleaned and re-sterilized.

As shown in FIG. 1, a sleeve 34 according to the invention may also be provided to temporarily maintain the head 12 on a neck extension 36 projecting from the proximal end 38 of the proximal body 16. In the illustrative embodiment, the head 12 includes a female junction element in the form of a tapered opening 13 and the neck extension 36 includes a male junction element in the form of a tapered shaft 37. The gender of these components may be reversed and still be within the scope of the invention. The sleeve 34 permits temporary firm seating of the head 12 on the neck extension 36 without locking the taper. Such 34 a sleeve may be used to temporarily fit modular components of a variety of types of orthopaedic implants together for joints throughout the body.

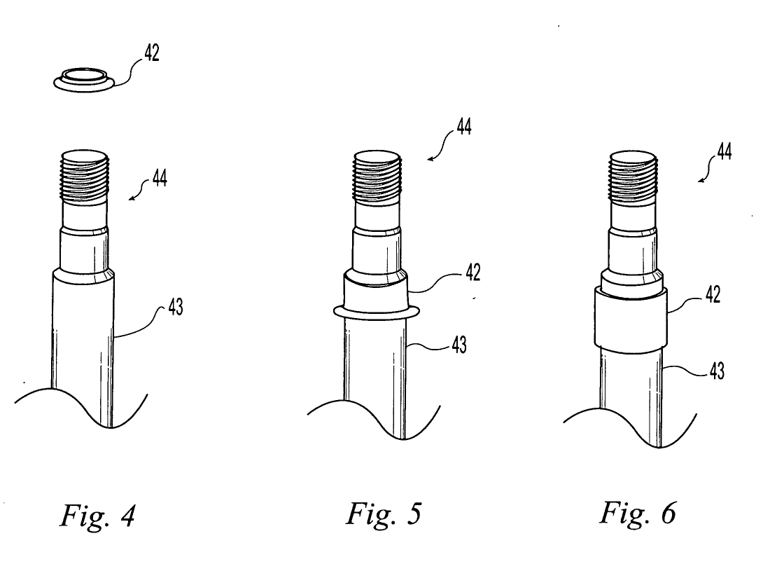

FIGS. 4-6 depict an alternate arrangement for deploying the sleeve of the present invention. In this arrangement, the sleeve 42 is provided in a rolled configuration. The sleeve 42 is positioned over the male side 43 of the modular junction and unrolled into position on the component 44. The sleeve 42 may stretch as it is unrolled so that it fits tightly on the component 44 or it may unroll to a substantially un-stretched shape. Trial fitting of proximal body components 16 may then proceed as described above.

It will be understood by those skilled in the art that the foregoing has described illustrative embodiments of the present invention and that variations may be made to these embodiments without departing from the spirit and scope of the invention defined by the appended claims.

1. A sleeve for use with a modular orthopaedic implant having a first component with a male junction element and a second component with a female junction element for receiving the male junction element to couple the components together, the sleeve comprising:
a hollow sleeve body having an outer portion able to be received in the female junction element and an inner portion able to receive the male junction element, the sleeve body being positionable between the male and female junction elements; and
means for temporarily maintaining the first and second components in an assembled condition.

2. The sleeve of claim 1 wherein the means for maintaining comprises friction engagement between the sleeve and each of the first and second components.

3. The sleeve of claim 1 wherein the sleeve is stretchable to tightly conform to the shape of the male junction element.

4. The sleeve of claim 1 wherein the sleeve is initially rolled into a ring shape and is able to be unrolled over the male junction element.

5. A sleeve for use with a modular hip stem having a stem component and a proximal body component, the stem component having a tapered male extension and the proximal body having a correspondingly tapered bore for receiving the extension, the sleeve comprising:
a hollow sleeve body having an outer portion able to be received in the tapered bore and an inner portion able to receive the tapered extension, the sleeve body being positionable between the male and female junction elements, the sleeve body engaging the implant components to temporarily maintain them in an assembled condition.

6. The sleeve of claim 5 wherein the sleeve body includes an outer taper and an inner taper.

7. The sleeve of claim 5 wherein the sleeve body is resilient and the inner dimension is initially smaller than the tapered extension but is stretchable to fit tightly over the tapered extension.

8. A combination of a sleeve and a modular orthopaedic implant, the combination comprising:
a modular orthopaedic implant having a first component with a male junction element and a second component with a female junction element for receiving the male junction element to couple the components together; and
a hollow sleeve having an outer portion able to be received in the female junction element and an inner portion able to receive the male junction element, the sleeve being positionable between the male and female junction elements; and
means for temporarily maintaining the first and second components in an assembled condition.

9. The sleeve of claim 8 wherein the first and second components are provisional implant components.

10. The sleeve of claim 8 wherein the first and second components are actual implantable components.

11. The sleeve of claim 8 wherein one of the first and second components is a provisional implant component and the other of the first and second components is an actual implant component.

12. The sleeve of claim 8 wherein one of the male and female junction elements comprises metal and the other of the male and female junction elements comprises a polymer.

13. The sleeve of claim 8 wherein the male and female junction elements both comprise metal.

14. The combination of claim 8 wherein the first component comprises a stem of a femoral hip implant and the second component comprises a proximal body of a femoral hip implant.

15. The combination of claim 14 further comprising a modular head component engageable with the proximal body and an acetabular component engageable with the head component.

16. The combination of claim 8 wherein the first component comprises a neck extension of a femoral hip implant and the second component comprises a head of a femoral hip implant.

17. A method of temporarily joining modular orthopaedic implant components, the method comprising:
providing a modular orthopaedic implant having a first component with a male junction element and a second component with a female junction element for receiving the male junction element to couple the components together;
providing a sleeve having a hollow sleeve body with an outer portion able to be received in the female junction element and an inner portion able to receive the male junction element; and
positioning the sleeve between the first and second components with the sleeve received in the female junction element and the male junction element received in the sleeve to temporarily maintain the first and second components in an assembled condition.

18. The method of claim 17 further comprising:
stretching the sleeve over the male junction element.

19. The method of claim 17 further comprising:
providing the sleeve in an initial rolled-up configuration; and
unrolling the sleeve over the male junction element.

20. The method of claim 17 further comprising:
ascertaining the fit of the first and second components with a patient's anatomy by placing the first and second components in a surgical opening in the patients body with the sleeve interposed between them to hold them in an assembled condition;
separating the first and second components while leaving the sleeve engaged with one of the components;
engaging another component with the sleeve to create another assembly; and
ascertaining the fit of the new assembly with the patient's anatomy.
